(19) United States
(12) Patent Application Publication
(10) Pub. No.: US 2022/0295907 A1
(43) Pub. Date: Sep. 22, 2022

(54) Sanitary Napkin Positioning Garment Assembly

(71) Applicants: Brandi Harris, Vallejo, CA (US); Kayla Powell, Vallejo, CA (US)

(72) Inventors: Brandi Harris, Vallejo, CA (US); Kayla Powell, Vallejo, CA (US)

(21) Appl. No.: 17/203,837

(22) Filed: Mar. 17, 2021

(51) Int. Cl. A41B 9/02; A61F 13/56

(52) U.S. Cl. CPC A41B 9/02; A61F 13/5605; A41B 2400/00

(57) ABSTRACT

A sanitary napkin positioning garment assembly for use during menstruation includes boxers, which comprises a waist opening and a pair of leg openings so that the boxers can be donned by a female user. Panties are positioned in and engaged to the boxers. The panties are resiliently stretchable and thus conform to a figure of the female user. A sanitary napkin can be attached to the panties so that the sanitary napkin is positioned over a vagina of the female user, for absorbing a flow of blood therefrom.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to garment assemblies and more particularly pertains to a new garment assembly for use during menstruation. The present invention discloses a garment assembly comprising integral panties configured to conform to a figure of female user, such that the panties are configured for attachment of sanitary napkin and for maintaining the sanitary napkin in position over a vagina of the female user.

### (2) Description of Related Art Including Information Disclosed Under 37 CFR 1.97 and 1.98

The prior art relates to garment assemblies. Prior art garment assemblies may comprise boxers intended for male users and having integral briefs, underpants, and the like, for supporting male genitalia. What is lacking in the prior art is a garment assembly comprising integral panties configured to conform to a figure of female user, such that the panties are configured for attachment of sanitary napkin and for maintaining the sanitary napkin in position over a vagina of the female user.

### BRIEF SUMMARY OF THE INVENTION

An embodiment of the disclosure meets the needs presented above by generally comprising boxers, which comprises a waist opening and a pair of leg openings so that the boxers are configured to be donned by a female user. Panties are positioned in and engaged to the boxers. The panties are resiliently stretchable and thus are configured to conform to a figure of the female user. The panties are configured for attachment to a sanitary napkin so that the sanitary napkin is positioned over a vagina of the female user, for absorbing a flow of blood therefrom.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a sanitary napkin positioning garment assembly according to an embodiment of the disclosure.

FIG. 2 is a top view of an embodiment of the disclosure.

FIG. 3 is a cross-sectional view of an embodiment of the disclosure.

FIG. 4 is a rear view of an embodiment of the disclosure.

### DETAILED DESCRIPTION OF THE INVENTION

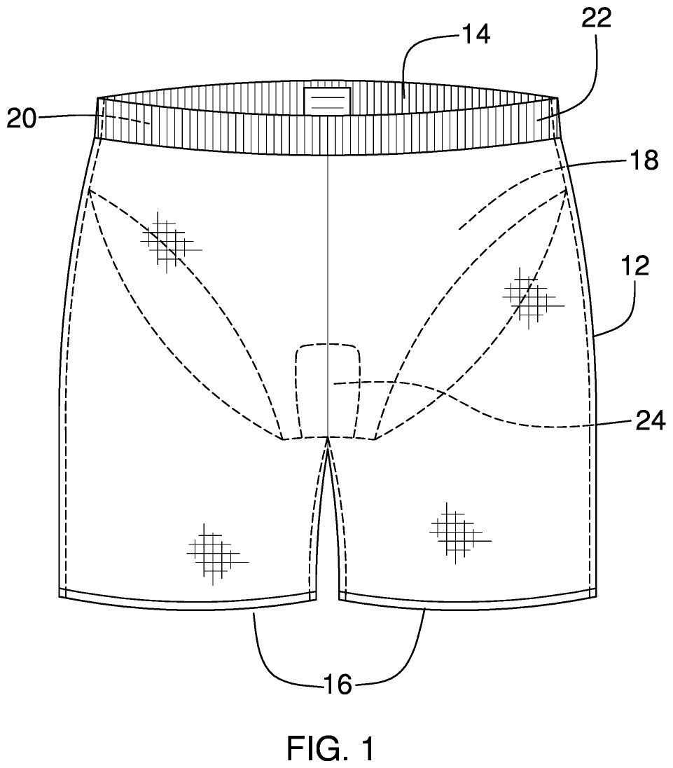

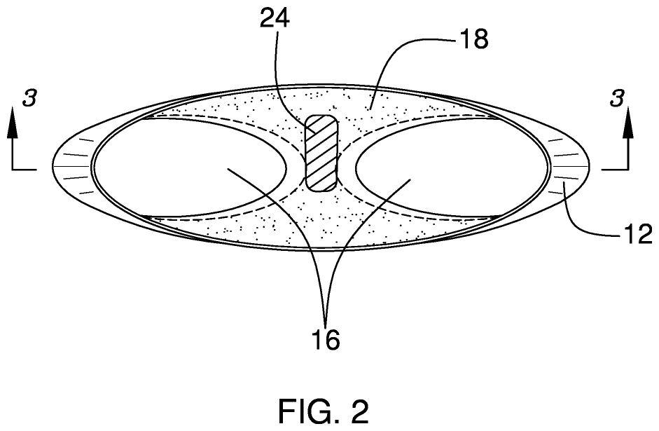

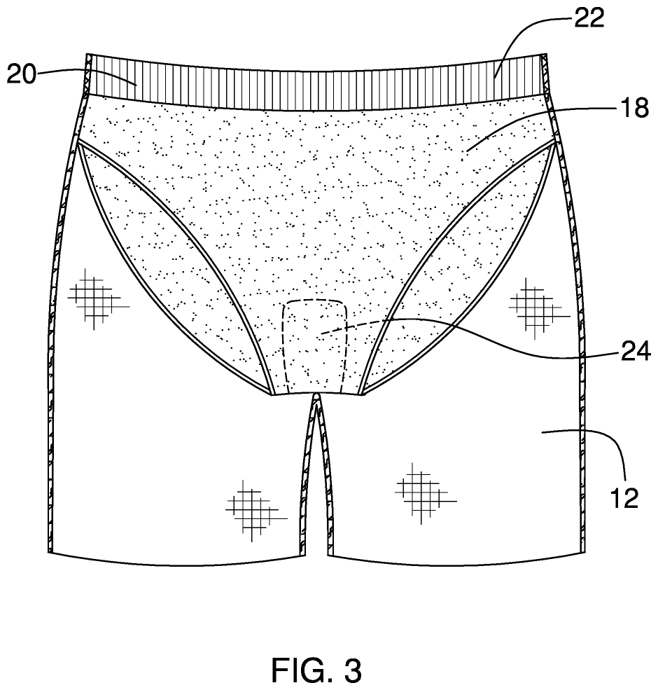

With reference now to the drawings, and in particular to FIGS. 1 through 4 thereof, a new garment assembly embodying the principles and concepts of an embodiment of the disclosure and generally designated by the reference numeral 10 will be described.

As best illustrated in FIGS. 1 through 4, the sanitary napkin positioning garment assembly 10 generally comprises boxers 12, which comprises a waist opening 14 and a pair of leg openings 16 so that the boxers 12 are configured to be donned by a female user. Panties 18 are positioned in and engaged to the boxers 12. The panties 18 are resiliently stretchable and thus are configured to conform to a figure of the female user.

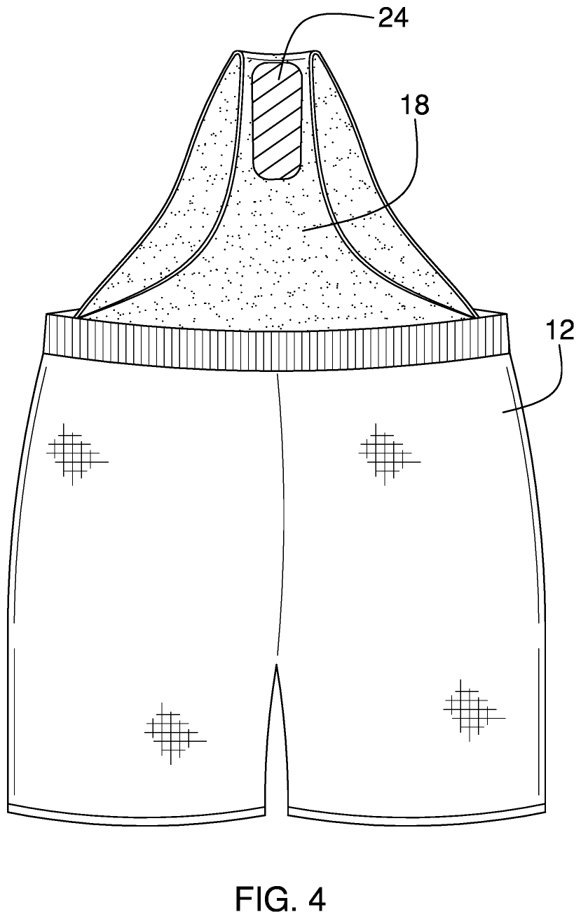

The panties 18 comprise cotton, lace, latex, leather, polyether-polyurea copolymer, mesh, nylon, polyvinyl chloride, polyester, rawhide, satin, silk, or a combination thereof. The panties 18 may be engaged by a waistband 20 thereof to a waistband 22 of the boxers 12, as shown in FIG. 4. The panties 18 are configured for attachment to a sanitary napkin 24 so that the sanitary napkin 24 is positioned over a vagina of the female user, for absorbing a flow of blood therefrom.

The assembly 10 is anticipated to be of use to female users of menstruating ages, and who desire the comfort of wearing boxer shorts. The assembly 10, wherein the panties 18 are integral to the boxers 12, allows female users the freedom of wearing boxer shorts while having an option of attaching a sanitary napkin 24 to the panties 18 should menstruation occur.

In use, the assembly 10 is donned by the female user. When needed, a sanitary napkin 24 can be engaged to the panties 18, such that the sanitary napkin 24 is positioned over a vagina of the female user to absorb a flow of blood therefrom.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sanitary napkin positioning garment assembly comprising:
boxers comprising a waist opening and a pair of leg openings, such that the boxers are configured for donning by a female user; and
panties positioned in and engaged to the boxers, the panties being resiliently stretchable, wherein the panties are configured to conform to a figure of the female user, wherein the panties are configured for attachment to a sanitary napkin, such that the sanitary napkin is positioned over a vagina of the female user for absorbing a flow of blood therefrom.

2. The sanitary napkin positioning garment assembly of claim 1, wherein the panties are engaged by a waistband thereof to a waistband of the boxers.

3. The sanitary napkin positioning garment assembly of claim 1, wherein the panties comprise cotton, lace, latex, leather, polyether-polyurea copolymer, mesh, nylon, polyvinyl chloride, polyester, rawhide, satin, silk, or a combination thereof.

4. A sanitary napkin positioning garment system comprising:
boxers comprising a waist opening and a pair of leg openings, such that the boxers are configured for donning by a female user; and
panties positioned in and engaged to the boxers, the panties being resiliently stretchable, wherein the panties are configured to conform to a figure of the female user; and
a sanitary napkin engaged to the panties, such that the sanitary napkin is positioned over a vagina of the female user, wherein the sanitary napkin is configured for absorbing a flow of blood therefrom.

5. The sanitary napkin positioning garment system of claim 4, wherein the panties are engaged by a waistband thereof to a waistband of the boxers.

6. The sanitary napkin positioning garment system of claim 4, wherein the panties comprise cotton, lace, latex, leather, polyether-polyurea copolymer, mesh, nylon, polyvinyl chloride, polyester, rawhide, satin, silk, or a combination thereof.

7. A sanitary napkin positioning garment assembly comprising:
boxers comprising a waist opening and a pair of leg openings, such that the boxers are configured for donning by a female user; and
panties positioned in and engaged to the boxers, the panties being resiliently stretchable, wherein the panties are configured to conform to a figure of the female user, wherein the panties are configured for attachment to a sanitary napkin, such that the sanitary napkin is positioned over a vagina of the female user for absorbing a flow of blood therefrom, the panties being engaged by a waistband thereof to a waistband of the boxers, the panties comprising cotton, lace, latex, leather, polyether-polyurea copolymer, mesh, nylon, polyvinyl chloride, polyester, rawhide, satin, silk, or a combination thereof.
